(19) United States
(12) Patent Application Publication
(10) Pub. No.: US 2009/0275850 A1
(43) Pub. Date: Nov. 5, 2009

(54) Electrocardiographic (ECG) Data Analysis Systems and Methods

(76) Inventors: Anil C. Mehendale, Brecksville, OH (US); Shichao Lei, Akron, OH (US); John P. Kroehle, JR., Hudson, OH (US)

Correspondence Address: FISH & RICHARDSON P.C., P.O. BOX 1022, MINNEAPOLIS, MN, 55440-1022, US

(21) Appl. No.: 12/113,006

(22) Filed: Apr. 30, 2008

(51) Int. Cl. A61B 5/0452; A61B 5/044

(52) U.S. Cl. 600/523; 600/509

(57) ABSTRACT

Computer-implemented methods, computer program products, and systems are disclosed for analyzing electrocardiogram (ECG) traces or other types of signals. In one aspect, a computer-implemented method of performing such an analysis on an ECG trace includes defining a match region within an ECG signal template comprising one cardiac cycle. The match region comprises a portion of the ECG signal template that is shorter than the entire template. The method also includes performing a similarity comparison analysis on an ECG trace. The similarity comparison analysis includes, for at least one cardiac cycle within the ECG trace, a comparison of the defined match region to a corresponding region of the cardiac cycle of the ECG trace to determine a degree of similarity between the defined match region and the corresponding region of the cardiac cycle of the ECG trace. The method also includes producing a results output based upon the similarity comparison analysis.

### TECHNICAL FIELD

This disclosure relates to the analysis of electrocardiographic (ECG) data or other similar periodic data.

### BACKGROUND

An ECG is a recording of electrical activity of a heart of a subject over time. ECG traces are often analyzed to identify characteristics of the subject heart rhythm, and how that may vary over time. One method of analyzing an ECG trace involves first defining a template of a cardiac cycle, and then comparing that template to cycles within the ECG trace to identify cardiac cycles that match the defined template to a specified degree of similarity.

There are various scenarios in which more useful and flexible ECG analysis systems would be useful. One such scenario is in the context of pharmaceutical drug testing, in which an ECG trace of a subject being given a drug may be acquired for analysis to determine how the subject's heart rhythm was impacted over time by the drug. In such a case, there may be many subject from which ECG traces are being acquired, and those ECG traces may be acquired over lengthy periods of time. As such, it can be seen that the amount of ECG data for analysis in such a scenario is very significant. This is just one example of an scenario in which large amounts of ECG data may be created for analysis. As such, powerful systems are needed to perform efficient and effective ECG analysis.

### SUMMARY

This document discloses computer-implemented methods, computer program products, and systems for analyzing electrocardiogram (ECG) traces or other types of signals. In one aspect, a computer-implemented method of performing such an analysis on an ECG trace includes defining a match region within an ECG signal template comprising one cardiac cycle. The match region comprises a portion of the ECG signal template that is shorter than the entire template. The method also includes performing a similarity comparison analysis on an ECG trace. The similarity comparison analysis includes, for at least one cardiac cycle within the ECG trace, a comparison of the defined match region to a corresponding region of the cardiac cycle of the ECG trace to determine a degree of similarity between the defined match region and the corresponding region of the cardiac cycle of the ECG trace. The method also includes producing a results output based upon the similarity comparison analysis.

Various implementations of the method may include one or more of the following features. The method may further include defining search window parameters used to identify the corresponding region of the cardiac cycle of the ECG trace that is compared to the match region. The search window parameters may include a specified cardiac event that is identified in the cardiac cycle by attribute analysis. The search window parameters further comprise a time period defined in relation to the cardiac event.

The defining of the match region in the method described above may include receiving user input that defines the match region. The user input that defines the match region may include user input that selects a marked cardiac event related to the defined match region. The user input that defines the match region may further include user input that selects a period of time in relation to the marked cardiac event. In alternative implementations, the defining of the match region may include receiving user input that selects a starting point and an ending point for the match region.

In the method described above, the defining of the match region may include defining a first match region and a second match region that is different from the first match region. In such a case, the performing of the similarity comparison analysis on the ECG trace may include performing the similarity comparison analysis a first time using the first defined match region and performing the similarity comparison analysis a second time using the second defined match region. The method described above may further include specifying which of the cardiac cycles within the ECG trace are to be included in the similarity comparison analysis.

The output provided by the method described above may be provided on a visual display device. The results output may include a total number of cardiac cycles processed in the similarity comparison analysis, and a total number of the processed cardiac cycles for which the corresponding region of the cardiac cycle was determined to have the specified degree of similarity to the match region. In addition or alternatively, the results output may include a time distribution of when there occurred, in the ECG trace, cardiac cycles for which the corresponding region of the cardiac cycle was determined to have the specified degree of similarity to the match region. In such a case, the results output may include histogram formatted results for each of multiple different time periods within the period of time during which the ECG trace was obtained. In addition, the results output may include, for each of the multiple different time periods, a total number of cycles processed in the similarity comparison analysis within the time period, and a total number of the processed cardiac cycles within the time period for which the corresponding region of the cardiac cycle was determined to have the specified degree of similarity to the match region. The output with the results may be provided on a visual display device.

The ECG trace under analysis using the method described above may be obtained from a subcutaneously implanted device that obtains a subcutaneous ECG trace and telemeters digital data comprising the subcutaneous ECG trace to external equipment for processing. Alternatively, the ECG trace is obtained using surface electrodes place on an external surface of a subject. Other signals to which the method may be applied include other biological signals such as action potentials which are pulse-like voltages that travel across a cell membrane, such as an axon of a neuron, cardiac muscle cells (in the case of what are commonly referred to as electrograms), and plant cells. Other signals that may be analyzed using these techniques are respiratory signals, including such signals during times of coughing or sniffing. In addition, the techniques may have applicability to other periodic or otherwise repeating signals for which template analysis techniques are useful, or in other words, where characteristics of a signal are being analyzed.

In various implementations of the methods described above, the method may further include defining a cycle of the ECG trace under analysis to serve as the ECG signal template. Alternatively, the ECG signal template may be a cardiac cycle taken from an ECG trace that is different from the ECG trace on which the similarity comparison analysis is performed.

In another more general aspect, there is provided a computer-implemented method of analyzing a signal trace, which includes defining a match region within a signal template. The match region includes a portion of the template that is shorter than the entire template. The method further includes performing a similarity comparison analysis on the signal trace. The similarity comparison analysis includes, for at least a portion of the trace having a time span that corresponds with the signal template, a comparison of the defined match region of the template to a corresponding region of the portion of the signal trace to determine a degree of similarity between the defined match region and the corresponding region of the signal trace. The method also includes producing a results output based upon the similarity comparison analysis.

In various implementations of this method, the signal trace may be an electrocardiogram (ECG) signal trace, and in such a case the signal template may be a cardiac cycle of an ECG signal trace. In other implementations, the signal trace may be an action potential trace, for example, an electrogram trace acquired using a lead that extends within a heart chamber of a subject. The method may further include defining search window parameters used to identify the corresponding region of the portion of the signal trace that is compared to the match region, and other variations as described previously and below.

In yet another aspect, there is provided a computer-implemented method of analyzing an electrocardiogram (ECG) trace, which method includes defining a match region within an ECG signal template comprising one cardiac cycle. The match region includes a portion of the ECG signal template that is shorter than the entire template. The method also includes defining search window parameters used to identify a comparison region within one cardiac cycle of an ECG trace. The comparison region includes a portion of the one cardiac cycle that is shorter than the entire one cardiac cycle. The method also includes performing a similarity comparison analysis on an ECG trace. The similarity comparison analysis includes, for at least one cardiac cycle within the ECG trace, a comparison of the defined match region to the comparison region of the cardiac cycle of the ECG trace to determine a degree of similarity between the defined match region and the comparison region of the cardiac cycle of the ECG trace. The method also includes producing a results output based upon the similarity comparison analysis.

In other aspects, computing systems and computer program products are provided for performing the computer-implemented methods described above and in the following more detailed description. Such computer program products are tangibly embodied in computer storage medium, for example, and include software program instructions that when executed by a processor perform operations for performing the computer-implemented methods. In the case of a computing system, such a computing system may include a match region definition component that defines a match region within an ECG signal template comprising one cardiac cycle, wherein the match region comprises a portion of the ECG signal template that is shorter than the entire template; a similarity comparison analysis component that performs a similarity comparison analysis on an ECG trace, the similarity comparison analysis comprising, for at least one cardiac cycle within the ECG trace, a comparison of the defined match region to a corresponding region of the cardiac cycle of the ECG trace to determine if there is a specified degree of similarity between the defined match region and the corresponding region of the cardiac cycle of the ECG trace; and an output device to which a results output of the similarity comparison analysis is provided.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a block diagram of an example ECG data acquisition and analysis system.

FIGS. 2 and 3 are block diagrams of two example ECG acquisition systems that may be used in the system of FIG. 1

FIG. 4 is a flow diagram of a computer-implemented method for analyzing an ECG trace.

FIG. 5 is a flow diagram of a computer-implemented method for analyzing an ECG trace, which focuses on operations of a set-up phase and illustrates an iterative nature of analyses that may be performed.

FIGS. 6A-6B are screen snapshots that show example displays that may be provided on a display device while the FIG. 5 method is being performed.

FIG. 7 is a flow diagram of a computer-implemented method of setting up an ECG trace analysis.

FIGS. 8A and 8B are screen snapshots that show example displays that may be provided on a display device while the FIG. 7 method is being performed.

FIGS. 8C and 8D are graphs that illustrate the search region and the template match region that are defined for the parameters shown in the displays of FIGS. 8A and 8B.

FIG. 9 is a flow diagram of an alternative ECG trace analysis setup method.

FIG. 10 is a flow diagram of an overall computer-implemented method for conducting an analysis of an ECG trace.

FIGS. 11A and 11B are screen snapshots that may be provided on a display device, showing results of an analysis of an ECG trace.

FIG. 12 is a flow diagram of a computer-implemented that may be used to create an ECG template.

FIG. 13 is a block diagram of an example computing system in which the systems and methods described in this document may be implemented.

Like reference symbols in the various drawings indicate like elements.

### DETAILED DESCRIPTION

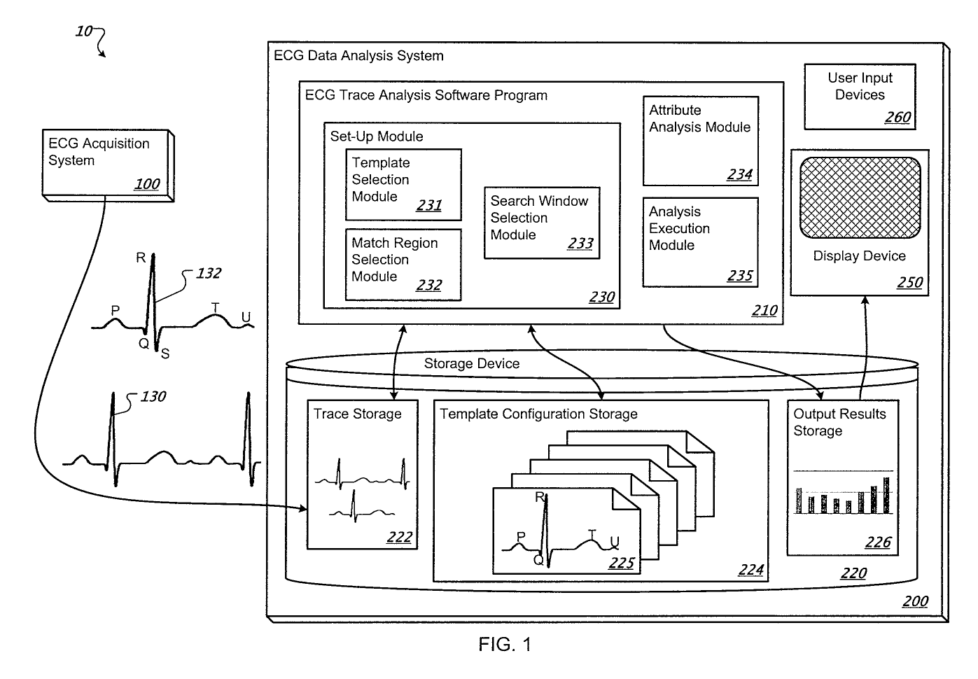

A biological signal acquisition and analysis system 10, shown in FIG. 1, may be used to acquire and analyze ECG data that reflect cardiac activity of a subject. The ECG data may be acquired from a surface of a subject, or using a subcutaneously implanted device. In other implementations, other types of data may be evaluated using techniques described in this document. For example, the techniques may be used in analyzing other biological signals such as action potentials which are pulse-like voltages that travel across a cell membrane, such as an axon of a neuron, cardiac muscle cells (in the case of what are commonly referred to as electrograms), and plant cells. Other signals that may be analyzed using these techniques are respiratory signals, including such signals during times of coughing or sniffing. In addition, the techniques may have applicability to other periodic or otherwise repeating signals for which template analysis techniques are useful, or in other words, where characteristics of a signal are being analyzed.

Generally, the system 10 shown in FIG. 1 includes an ECG data acquisition system 100 that acquires data from one or more subjects being monitored, and an ECG analysis system 200 that can receive the acquired ECG data and analyze the data. The system 10 is particularly well suited to analyze large amounts of ECG data (including, for example, numerous cardiac cycles) recorded over a period of time of several hours, days, or even longer. As one example, ECG data may be acquired from an animal subject undergoing pharmaceutical drug testing. In such a case, the data analysis system 200 may be used to study the acquired ECG data to understand the impacts of a drug on the subject's cardiac activity over time. The analysis system 10 is also well suited for analyses of ECG data taken from human subjects, under various different scenarios.

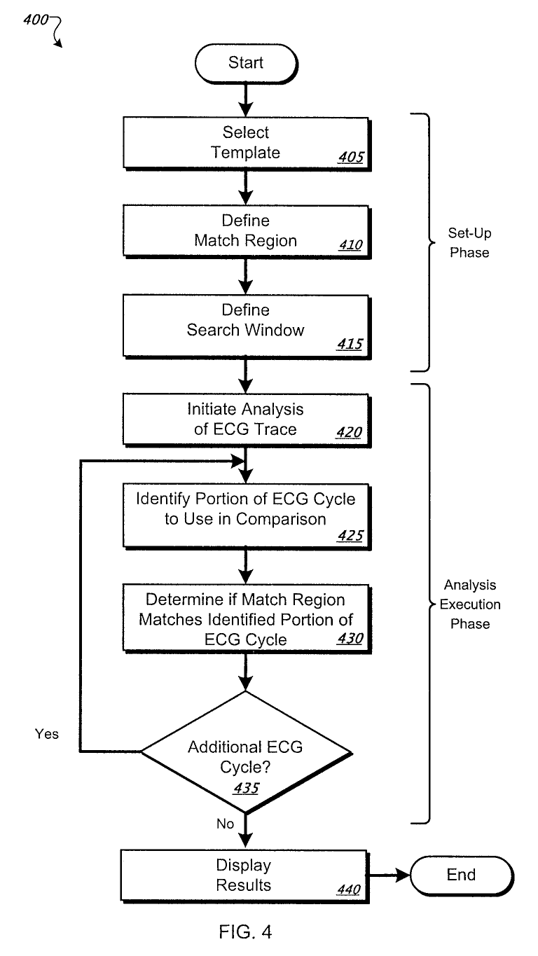

Generally in operation, the ECG data analysis system 200 is used to analyze an acquired ECG trace that may include many cardiac cycles. In one example, shown in FIG. 4, there is a set-up phase in which set-up functions are performed in advance of an analysis being performed on an ECG trace, and an execution phase when the analysis of an ECG trace is actually run. In the set-up phase, a user may, for example, first select one cycle from the acquired ECG trace to serve as a template for a similarity comparison that will be performed on other cardiac cycles in the ECG trace, as shown by operation 405.

The user then may, in operation 410, define a portion of the selected cardiac cycle (that is, the template) to be used in the similarity comparison. The defined portion of the template may be referred to as a match region. The selection of only a portion, and not all, of the template to serve as the match region may be useful, for example, because a user may wish to focus the ECG analysis only a specific selected portion of the cycle, for example, the P-wave, the QRS complex, the T-wave, etc. Alternatively, it may be useful for a user to observe that while the QRS complex portion of cycles in an ECG trace match a corresponding portion of a template, the T-wave portion of the cycles in the ECG trace do not. In addition, for T-waves in particular, important variations may be on a very small microvolt scale. As such, if an entire ECG template were compared to an entire ECG trace, differences between the T-waves could go unnoticed as they may be small when compared to the entire cardiac cycle. In this case, a transient change in the QRS complex could overwhelm a potential match in the T-wave, thus leading to the loss of a potential match. As such, it may be advantageous for the user to define a match region in the template that includes the T-wave and compare only the match region to an ECG trace when looking for a potential match.

After the match region of the template is selected in the FIG. 4 method, in operation 415 the user may set parameters for a search window that will be used to determine the portion of each cycle of the ECG trace against which the match region will be compared. It may be advantageous to limit the search window so as to limit the amount of computational time required to complete comparisons and/or to minimize false positives by defining specific portions of the ECG data to compare to the templates. For example, a user may be able to define a search window surrounding the T-waves in an ECG trace. When making a comparison, the match region in the template can be compared to the search windows in an ECG trace for the purpose of identifying a match.

To set search window parameters, the user may first select a cardiac attribute of the ECG cycle (for example, the T-wave, which would be appropriate in a case where the selected match region bounds the T-wave of the template), and then define a window of time in relation to the selected attribute. The user may also perform other set-up tasks not shown in FIG. 4, including, for example, identifying which of the cycles of the ECG trace are to be included in the similarity comparison analysis (for example, certain ones of them, all of them, etc.).

Referring still to FIG. 4, with the system 200 thus set up, the system 200 may then perform a similarity comparison analysis, or in other words, start the execution phase of the analysis. This begins with the analysis of the ECG trace being initiated, as shown in operation 420, which may be triggered, for example, by a user input to start the analysis. The system 200 then compares the configured match region to each of the cardiac cycles of the ECG trace that were defined by the user to be included in the analysis. For each such cardiac cycle being analyzed, the system first, as shown in operation 425, identifies the relevant portion of the cardiac cycle to be used in the comparison, as determined by the search window parameters that were set up. To identify the relevant portion of the cardiac cycle to be used, the system 200 first identifies, using attribute analysis techniques, where in the cycle the selected cardiac attribute occurs, and then, using the defined time-period search window parameters, identifies the portion of the cycle to use in the comparison.

Then, in operation 430, the system 200 makes a comparison of the match region (of the template) to the identified portion of the cardiac cycle (of the ECG trace) being analyzed. A match of the two may be identified when the comparison analysis determines that the match region has a specified degree of similarity to the identified portion of the cardiac cycle being analyzed. The comparison analysis may be done, for example, by scanning the match region through the identified region of the cycle being analyzed and at several locations in the scan (for example, at every sample point of the trace) determining if there exists the specified degree of similarity in the traces. In alternative implementations, such as implementations in which the match region of the template is longer than the comparison region of the trace under analysis, the comparison analysis may be done by scanning the comparison region through the match region in a similar manner.

The analysis may continue cycle-by-cycle until all of the cardiac cycles of the ECG trace are analyzed in this manner (or at least all of the cycles that were configured to be analyzed). This is indicated in the FIG. 4 flowchart by operation 435, which indicates that if an additional cycle is to be analyzed, processing returns to operation 425. After all of the cardiac cycles are analyzed, the system 200 may then, at operation 435, produce an output of this analysis. The output may, for example, identify the number of cycles for which a match was determined to exist, and information regarding where those cycles occurred. This may be displayed, for example, in histogram or other useful graphic form, as will be described in more detail later.

A user may then review the output of the analysis, and perform additional analyses of the ECG trace as appropriate. In fact, in many analyses the method of FIG. 4 may be repeated several times, using different templates, match regions, and search window parameters. By way of example, the user may determine from the output of a first analysis that the analysis was not useful, and may want to use a different cardiac cycle as the template, a different match region of the template, different search window, and/or different search parameters. In this case, the user may make new selections and run another analysis. As will be appreciated, the user may make these changes in the set-up very easily, and run different or additional analyses. In addition, a user may define several different match regions for multiple templates, and run an analysis that goes through each of the match regions in turn. In such a case, the system may determine which of several match regions most closely resemble cardiac cycles in the ECG trace being analyzed.

In a typical scenario, the user may determine, after running an analysis that produced useful results, that it would be further useful to run an additional analysis using a cardiac cycle in the ECG trace found to have different characteristics than the match region of the cardiac cycle used in the first analysis. In this case, the user may run another analysis using a different cardiac cycle as the template, or may use a different match region of the same template. This may be useful, for example, in a pharmaceutical drug testing scenario where there may be time periods of the ECG trace where the cardiac cycles match better with the first match region, while other time periods of the ECG trace match better with the second match region. As an example of where the user may decide to run an additional analysis using the same template as in the first analysis, but with a different match region, the user may select the T-wave region for the first analysis, and the QRS complex for the second analysis.

Further yet, templates and settings (match region and search window) derived from one ECG trace may be stored and used later in the analysis of another ECG trace. This may be useful, for example, where an ECG trace is taken from the same subject but during two different time periods. This approach may also be useful if there is sufficient similarity in the cardiac cycles—both normal and abnormal cycles—of two different subjects. In some cases, the positioning of electrodes may impact the usefulness of templates taken from one ECG trace in the analysis of another ECG trace. For example, if electrodes are positioned in different places in one acquired trace compared to another acquired trace, then templates taken from the first trace may not be useful in an analysis of the second trace. In a case of electrodes that are implanted subcutaneously, and thus are in the same position for multiple acquired traces, then the likelihood is greater that templates from one ECG trace will be usable in an analysis of another ECG trace from the same subject and the same implanted electrodes.

Referring back to FIG. 1, the example ECG analysis system 200 shown there includes an ECG trace analysis software program 210, memory storage device 220, and user interface devices including input devices 260 (keyboard, mouse, etc.) and a visual display device 250. The ECG analysis system 200 is a computing system that may take a variety of different forms, for example, a stand-alone computer and/or a laptop device with various peripheral devices, and/or a networked computer that makes use of servers and other networked computing resources.

The ECG analysis program 210 includes executable computer software instructions stored in an information carrier (for example, computer memory), and that are executed to enable the system 200 to perform various programmed functions. In the FIG. 1 example, the ECG analysis program 210 is made up of various software program modules, including, firstly, a set-up module 230, which allows a user to perform set-up tasks for an analysis of an ECG trace. Set-up tasks may include selecting a cardiac cycle of an ECG trace to serve as a template (performed by template selection module 231), specifying a match region of the template (performed by match region selection module 232), specifying a search window to be used in the analysis (performed by search window selection module 233), as well as other functions that will be described later.

The ECG analysis software program 210 also includes an attribute analysis module 234, which analyzes an ECG trace and identifies specific attributes of the ECG trace, such as an R-wave of a cardiac cycle, the T-wave of the cycle, etc. This module 234 may be used, or called by, the set-up module 230, for example to identify a cardiac cycle of an ECG trace. This may be done to enable the selection of cycles to serve as templates. In addition, the attribute analysis module 234 may identify other attributes in a cardiac cycle, such as the P-wave, T-wave, etc., which may also be useful in performing set-up tasks. The attribute analysis module 234 may also be used, or called by, an analysis execution module 235.

The attribute analysis module 234 can analyze ECG data, and identify individual ECG and/or cardiac events (e.g., the location of T-waves, the location of P-waves, the location of the QRS complex, and the like) within the ECG data. Individual cardiac cycles of an ECG trace can be stored in the trace storage 222 and/or used by the set-up module 230, along with user input, to create one or more ECG templates, which can be stored in a template configuration storage 224 included in the storage device 220. These templates can be accessed later by, for example, the analysis execution module 235 and used in the analysis of an ECG trace.

The analysis execution module 235 is the mechanism, in the FIG. 1 embodiment, that performs the analysis of an ECG trace cycle-by-cycle once the analysis has been set up. The analysis of a specified ECG trace may be performed in accordance with how the analysis was set up by the set-up module 230. The analysis execution module 235 may make use of the attribute analysis module 234, for example to identify a portion of each cardiac cycle in the trace against which the match region will be compared. In addition, the analysis execution module 235 makes the comparison of the template match region against the identified portion in each cardiac cycle, and may also produce an output of the analysis results.

The memory storage device 220 includes one or more computer storage devices capable of storing electronic files and data. These computer storage devices may be located physically near one another, or in other locations in a distributed system. The memory storage device 220, in the FIG. 1 example, includes storage 222 for ECG traces received from the ECG acquisition system 100. This enables ECG traces to be retained for later review, and to perform multiple analyses on the same ECG trace, as described above. There may be many ECG traces stored in the ECG trace storage 222, from many different subjects and many different time periods. In a typical scenario, ECG traces (e.g., the ECG trace 130 shown in FIG. 1) sent by the ECG acquisition system 100 are received by the ECG analysis system 200, stored in the analysis system's trace storage 222, and accessed by operation of the system 200 executing the ECG analysis program 210.

The memory storage device 220 also includes template configuration storage 225, which may include an templates and their configurations previously set up. The template configuration storage 225 may include the templates themselves (cardiac cycles), and may also include a user-specified match region for the template, a search window to be used in connection with the template, and/or other parameters related to the template such as a match threshold that may be used in a comparison analysis using the template. The memory storage device 220 also includes analysis output results storage 226, which includes files containing the results of analysis, including any set-up parameters for those results.

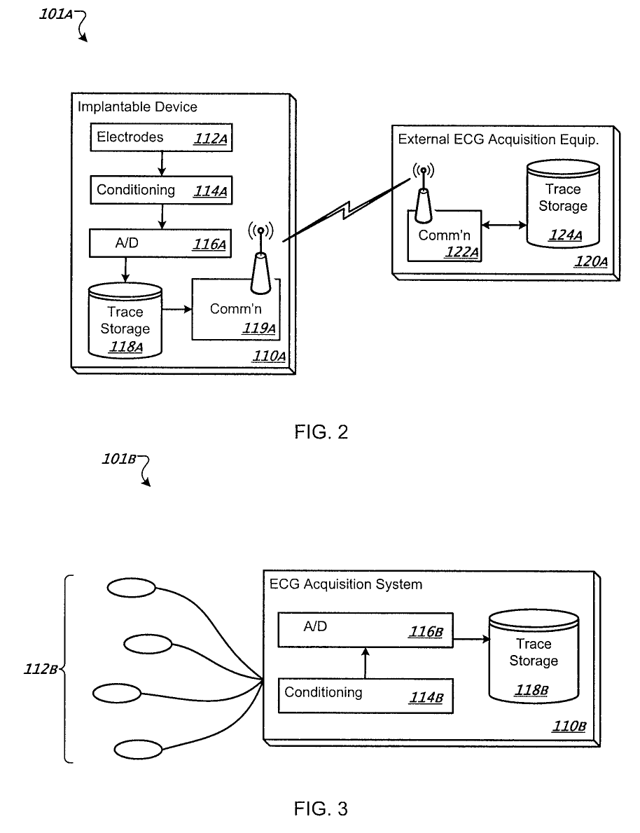

Referring again to the ECG acquisition system 100, this system may take a wide variety of different forms, depending on the types of analyses that are being done. FIG. 2 is an example acquisition system 101A where implanted electrodes are used. FIG. 3 is an example acquisition system 101B where surface, or skin, electrodes are used. Other signal acquisition systems are contemplated depending on the type of signal, including action potential signals, electrogram signals, etc.

Referring to FIG. 2, the ECG acquisition system 101A shown there includes an implantable device 110A and external equipment 120A. The implantable device 110A senses ECG signals from a position internal to the subject, and the external equipment 120A receives ECG data transmitted by the implantable module 110A. The implantable module 110A includes one or more electrodes 112A to sense ECG signals. These electrodes 112A may take a variety of different forms. For example, the electrodes 112A may be subcutaneous electrodes that extend from an implantable housing or that are integrated into the housing of the device 110A. In another example, the electrodes 112A may be electrodes associated with an endocardial lead. In this case, the ECG signal being acquired is an endocardial ECG signal, sometimes referred to as an electrocardiogram, EGM or Egram.

The implantable device 110A may also, but not necessarily, include conditioning circuitry 114A for conditioning ECG signals sensed by the electrodes 112A, analog-to-digital converting circuitry (A/D converter) 116A, memory storage device 118A to store data related to the ECG signals (e.g., an ECG trace), and wireless communication circuitry 119A (radio frequency, optical, etc.) to transmit the ECG trace to the external equipment 120A. Although a wireless embodiment of communication device 119A is shown in FIG. 2, it will be appreciated that in other scenarios there may be a wired connection between the internally implanted electrodes and the external equipment.

To briefly describe the operation of the FIG. 2 implantable device 110A, ECG signals are sensed by the electrodes 112A, and then conditioned (e.g., noise filtered, and the like) by the conditioning circuitry 114A. An output of the conditioning circuitry 114A is then converted from analog form to digital form by the A/D converter 116A, and the digital data are stored in the memory storage device 118A until the data are transmitted to the external equipment 120A. In some scenarios, the implantable device 110A may transmit the ECG data as soon as it is acquired, while in other scenario, the implantable device 110A may transmit all the ECG data of a trace after the entire trace has been acquired, or may transmit ECG data in periodic short bursts.

The external equipment 120A includes a communication device 122A to receive ECG data, and a storage device 124A. In some embodiments, the external acquisition module 120A may send a request to the implanted device 110A, instructing the implanted module 110A to send, via the communication devices 119A and 122A, an ECG trace to the external module 120A. When the request is received by the implanted module 110A, an ECG trace stored within the storage device 118A can be transferred from the implanted module 110A to the external module 120A via the communication devices 119A and 122A and stored in the storage device 124A. Data (e.g., a digitized ECG trace 130 as shown in FIG. 1) can subsequently be transferred from the external module 120A to the ECG data analysis module 200.

Referring to FIG. 3 now, the other example ECG acquisition system 101B can measure external ECG signals of a subject. The acquisition system 101B includes an ECG acquisition system 110B and one or more surface, or skin, ECG electrodes 112B. The acquisition system 110B, in this implementation, includes conditioning circuitry 114B, analog to digital converting circuitry (A/D converter) 116B, and a storage device 118B to store data related to the biological signals. For example, the ECG acquisition system 101B can be an external device that can obtain one or more sets of real time ECG data from the subject via the surface electrodes 112B positioned externally to a subject. These signals can be conditioned by the conditioning circuitry 114B and converted from analog signals to digital data by the one or more A/D converters 116B. The digital data (e.g., the digitized ECG trace 130 as shown in FIG. 1) can then be stored in the storage device 118B until the data is transferred to the analysis module 200.

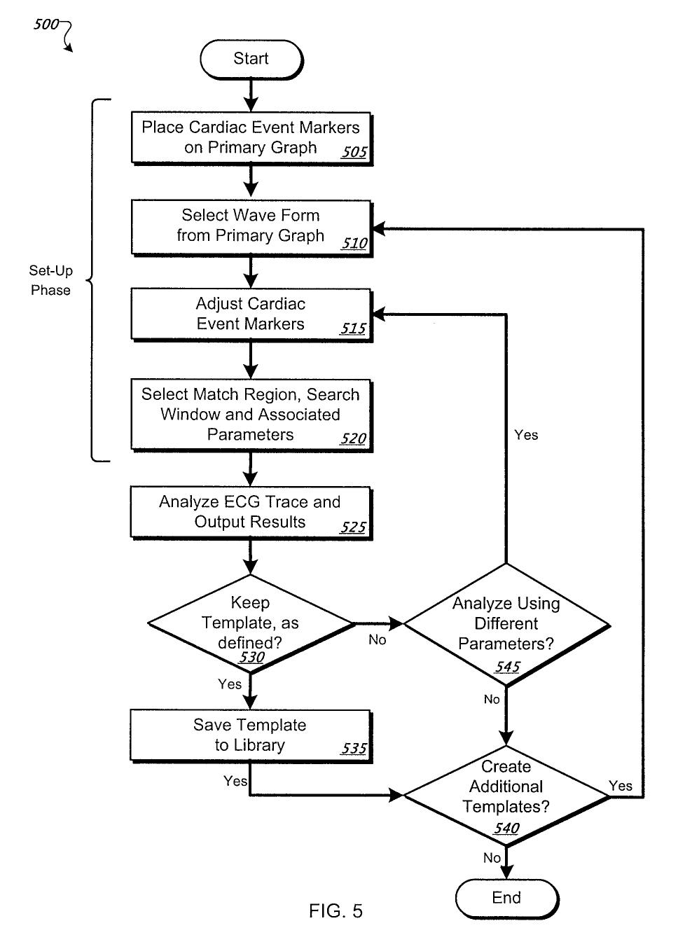

FIG. 5 shows an example method of the set-up phase of an ECG trace analysis method, and also shows how in a common scenario of ECG trace analysis there may be multiple, different, and iterative analyses executed upon a single ECG trace. The method of FIG. 5, in one implementation, involves operations by the ECG data analysis system 200 of FIG. 1, and in particular the execution by a processor of software instructions, such as software instructions of the ECG analysis program 210. When referring below to operations being performed by modules of the software program 210, it will be appreciated that those modules are components that include a processor executing software instructions.

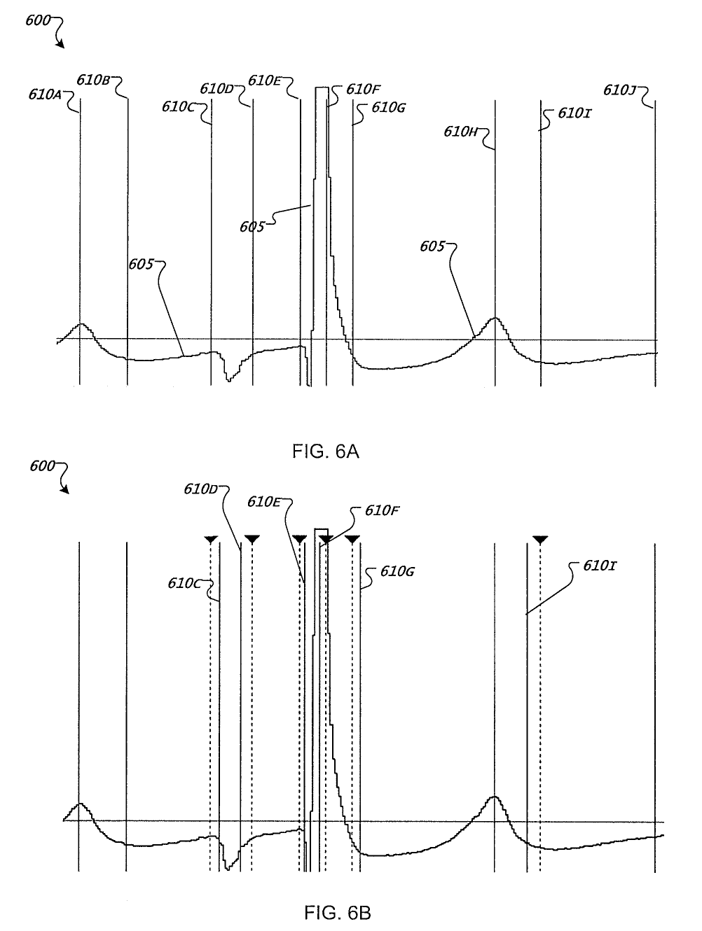

Before the ECG analysis method of FIG. 5 begins, an ECG trace will have been selected and obtained or retrieved, for example from trace storage 222 (FIG. 1), and before that from the ECG acquisition system 100. In operation 505 of the FIG. 5 method, the attribute analysis module 234 analyzes the selected ECG trace and identifies where in the ECG trace various cardiac events occur. For example, the system may identify cardiac events such as the “P”, “Q”, “R”, “S”, “T”, and “U” waves of a cardiac cycle, and can mark the location of when those events occur. As will be described in more detail in connection with FIGS. 6A and 6B, a primary graph of an ECG trace can be displayed, with cardiac event markers indicated on the displayed ECG trace at appropriate locations. Such a display may be provided on the system's display device 250 (FIG. 1). In typical implementations, users will be able to navigate and display, or zoom in on, various portions of the ECG trace, so as to view cycles that may be selected to be a template, and view where events in those cycles have been determined to occur, as indicated by displayed markers. Such a display may be provided on the system's display device 250 (FIG. 1).

Next, in operation 510, a user can cause the template selection module 231 to select a portion of the ECG trace or wave form (e.g., select an individual ECG cycle) to use as a cardiac template. This may be done in a variety of ways, for example, by the user navigating to display a certain cardiac cycle of the trace, and then performing an input action to select the cycle, for example by a mouse click or some other means. In operation 515, the cardiac event markers determined in operation 505 can be adjusted in the template selection module 231, with input supplied by the user via the input devices 260 and parameters. For example, a marker originally placed by the attribute analysis module 234 indicating the end of the T-wave can be moved, by a user, to earlier or later in the cardiac cycle.

The system allowing a user to adjust the location of a marker may be useful, because there may not be a consensus between different researchers as to where a cardiac event occurs, and so different researchers may mark the event in different places. In this regard, while there may be general agreement as to a proper location for the R-wave peak, because it is relatively clear and distinct, there may be more differing views as to the correct location for less distinct marks, for example, the end of the T-wave. Because in many implementations the ECG trace analysis system makes use of these markers are positioned on the template to define not only the match region of the template, but also the region within a cardiac cycle being analyzed to which the match region will be compared, it is typically important to allow a user to define where these markers are placed.

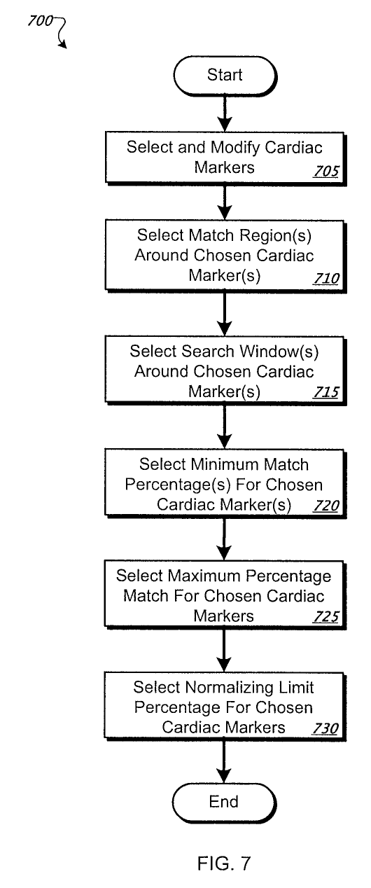

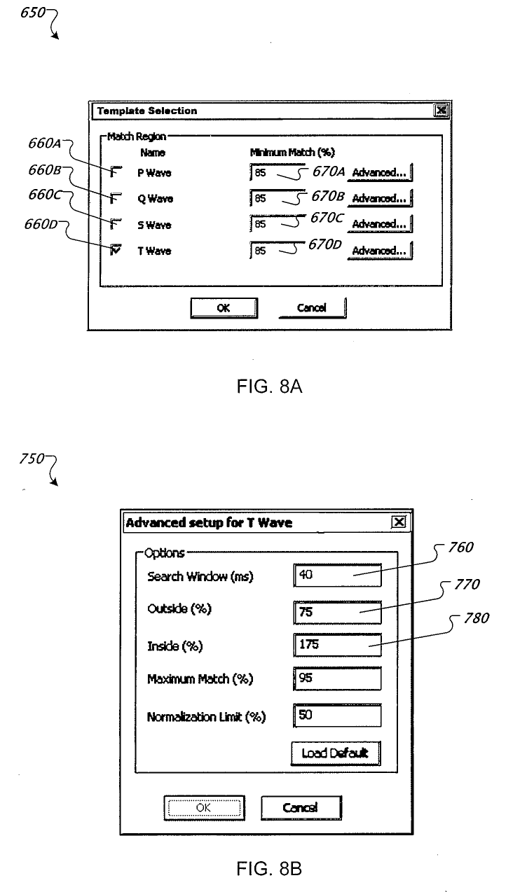

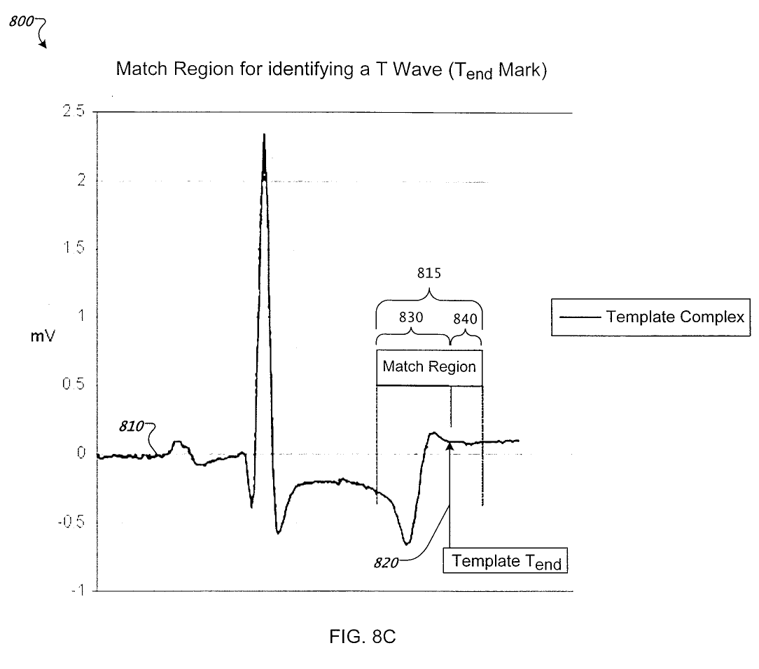

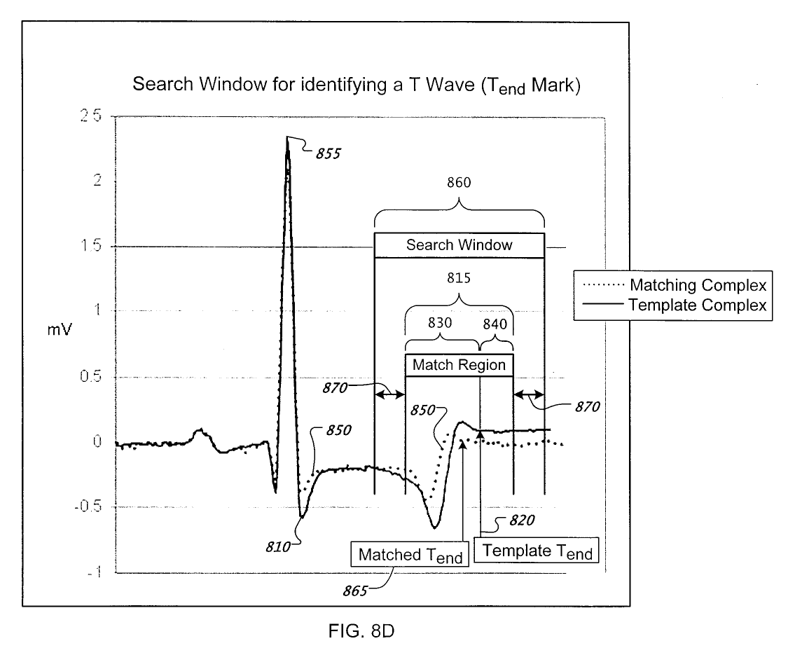

In operation 520, one or more match regions, corresponding search windows, and associated parameters used in a comparison of an ECG trace to ECG templates are selected by a user. An example of how this may be done is discussed in more detail in connection with FIGS. 7, and 8A-8D. With respect to the selection of a match region, a user may, for example, wish that a portion of the template focused on the T-wave of the cardiac cycle serve as the match region. In such a case, the user would initiate an input that selects that portion of the template. In addition to that input, various other user inputs may be provided to carry out the functions of operation 520 of the FIG. 5 method, including inputs that specify the boundaries of the match region as measured in relation to an identified event (e.g., the peak and the end of a T-wave in the case of a T-wave match region), inputs that specify the portion of a cycle to which the match region will be compared (for example, search window specifications), and inputs that specify the degree of similarity needed in the comparison between the template and a portion of a cardiac cycle for the comparison to be considered a match. Various other parameters may also be input as part of operation 520, as will be discussed later and as will be understood by those of skill in the art.

In operation 525, the execution of an analysis of the ECG trace is conducted, and results of the analysis are produced. In one example, this operation may be performed by the analysis execution module 235 that compares each of the specified cardiac cycles of the ECG trace (for example, all of the cardiac cycles of the trace if they are all specified to be analyzed) to the ECG template. The results of the comparison analyses of all the cycles may then be output and stored in memory 220 (FIG. 1) as a results file 226, and/or transmitted to the display device 250 to provide a visual display of the results to the user.

The analysis execution module 235 can also allow a user to select certain parameters associated with the analysis. For example, a user can supply the analysis execution module 235 with information, such as, the range of data in an ECG cycle that should be analyzed, if all cycles or only previously unmatched cycles should be analyzed, if the entirety of each individual ECG cycle within the ECG trace should be analyzed, or only region(s) within the cycles (e.g., search regions). The analysis execution module 235 can determine the percentage of individual ECG cycles within an ECG trace that match an ECG template according to the parameters selected during operations 515, 520, and/or 525. The analysis execution module 235 can also make an indication (e.g., by an arrow, a change in color, a highlighted region, and the like) on a graph of the ECG trace as to which ECG cycles in the ECG trace match a template. In operation 530, the user can make a determination, based at least in part on the results of the analysis, whether the template 225 should be saved. In some embodiments, it may be desirable for a template to match, at a minimum, a certain percentage (e.g., 5%, 17%, 40%, 56%, or the like) of ECG cycles within an ECG trace. If the template, including parameters, is acceptable to the user and should be kept, the process 500 can execute operation 535 and the template as defined can be saved to the template configuration storage 224.

In some embodiments, it may be desirable to continue to analyzed different match regions and templates until there is found a template that matches a certain percentage of the cycles in a trace (e.g., 47%, 86%, 90%, or the like), or greater, of the individual ECG cycles within an ECG trace match one or more ECG templates. In operation 540, if it is determined that that no more match regions or templates need to be analyzed, the process 500 can terminate. If, however, it is determined that more templates and/or match regions are to be analyzed, the method 500 may return to operation 510, where the user can select an additional wave form (e.g., an individual ECG cycle 132) from the primary graph of the ECG trace to use in the creation of an additional ECG template. Alternative, the method 500 may proceed with an analysis using the next match region that has already been set up.

Further with respect to operation 530, if a user determines that the template, as currently defined, should not be kept, operation 545 is performed and the user can make a determination if the same template should be modified and used in a subsequent analysis. In addition, instead of a user making these determinations, and in a case where several match regions are configured before an analysis is started, the system may make the determination as to whether to go on to another match region and do the analysis using that match region, or to terminate the processing because, for example, a sufficient number of the cycles in the trace have been matched to some match region.

If it is determined by the user, at operation 545, that the current template should be modified and reused in an analysis, the process 500 can return to operation 515, where parameters, such as the locations of the event markers and the match regions(s), can be adjusted. If the user determines, in operation 535, that no further modification to the template should be made, the template 225 can go unsaved and the process 500 can move to operation 540 to determine if additional templates are to be created.

Referring now to FIGS. 6A and 6B, two screen snapshots 600 and 620 are shown of one cardiac cycle 605 of an ECG trace. These screen snapshots may be displayed when a user is setting up the analysis, for example, during operation 515 of the FIG. 5. These screen snapshots illustrate how a user is able to adjust locations for events in the cardiac cycle (for example, earlier or later in time), from locations determined by an attribute analysis module.

In FIGS. 6A and 6B, determined cardiac events are indicated on the displays by vertical-line markers 610A through 610J. FIG. 6A shows the locations of the cardiac events as determined automatically by an attribute analysis module, and FIG. 6B shows some of the markers having been moved by user input.

In more detail, the FIG. 6A display has the following ten vertical-line markers indicated on the cardiac cycle 605: the peak of the T-wave from a preceding cardiac cycle (marker 610A), the end of the T-wave of the preceding cardiac cycle (610B), the beginning of a P-wave (610C), the end of the P-wave (610D), the Q-wave (610E), the peak of an R-wave (610F), the S-wave (610G), the peak of the T-wave (610H), the end of the T-wave (610I), and the beginning of the P-wave 605 for the next cycle (610J). By using an input device such as device 260 (FIG. 1), a user is able to cause the template selection module 231 to modify the location of any of the cardiac markers (e.g., by an on-screen drag and drop method, entering an offset, or the like).

FIG. 6B shows the display 600 graph of the ECG trace 600 after the user has made adjustments to the locations of six of the ten event markers, in particular markers 610C through 610G and 610I. Dashed vertical lines with an arrow on top indicate the original positions for the markers that were moved. To get from the display of FIG. 6A to the display of FIG. 6B, the following movements were made of the markers: marker 610C was moved later in time, marker 610D was moved earlier in time, marker 610E was moved later in time, marker 610F was moved earlier in time, marker 610G was moved later in time, and marker 610I was moved earlier in time. The other markers were not moved. Although not shown in the scenario depicted in FIGS. 6A and 6B, the system may be programmed so that a movement of one marker in time will cause other markers to be moved similarly and automatically in the same direction and amount of time.

Referring now to FIG. 7, there is shown a flow chart of a method 700 of an example set-up phase for an analysis, focusing on the definition of the match region in the template, the search window, and other set-up parameters (see operation 520 of FIG. 5). FIGS. 8A-8B show example screen snapshots that may be displayed on a display device for a user while the method 700 of FIG. 7 is being performed, and FIGS. 8C-8D illustrate the match region and search window settings shown in FIGS. 8A-8B. It will be appreciated that the operations of the method 700 shown in FIG. 7 need not be performed in the order specified in the flow chart, as parameters for the analysis can be selected or defined for an analysis in any order. In addition, it will further be appreciated that for the FIG. 7 method 700 default values may be used or suggested by an ECG analysis system, and thus a user may not perform an action to make the selections shown in the flow chart, but rather the system may make the selections indicated, for example, by accepting default values.

Before the method of FIG. 7 would begin, an individual ECG cycle typically would have already been selected to serve as a template, as described previously in connection with FIG. 5. The method of FIG. 7 includes operation 705, in which a user has the option to select and modify, as desired and appropriate, the location of various marked cardiac events in the selected cardiac cycle being used as the template. This may be done, for example, as was described previously in connection with the FIG. 5 method (operation 515) and the using the displays shown in FIGS. 6A and 6B.

In operation 710, the definition of a match region (or multiple different match regions), a match region for the template is defined. This operation includes a selection of a marked cardiac event. This may be done, for example, by a user making a selection of one of multiple different waves (P-wave, Q-wave, etc.) of a cardiac cycle, where the wave has a cardiac event associated with it. In such an example, the match region is defined in relation to a cardiac event, and may include a specified time period defined in relation to that cardiac event. In one example, the time period may include a period of time before the cardiac event and a period of time after the cardiac event.

In other implementations, a cardiac event may define the beginning of a match region (with a defined period of time after the beginning event), or alternatively, the end of a match region (with a defined period of time before the ending event). In still other implementations, the match region need not be defined in relation to a cardiac event of the template, but rather may simply be a defined portion of the template, with a starting point and an ending point. In addition, multiple match regions may be defined for a template in the set up, and multiple analyses may be run, with each one using a different defined match region.

The method 700 also includes an operation 715 in which a search region window is defined for use with a specific match region. The search window is used in a comparison analysis to identify a region of cardiac cycle against which the match region of the template is to be compared. Different match regions for a template may have different defined search regions. In an implementation in which the match region is defined in relation to one or more cardiac events in the template (as described above), the search window may be defined in relation to the same cardiac event type, and that event will be located during analysis execution. In addition, the definition of the search window may include a specification of the time period to be included in the search window that is defined in relation to the cardiac event, for example, periods of time before and after the cardiac event. As was the case with defining match regions in templates, a cardiac event may alternatively define the beginning of a search window (with a defined period of time after the beginning event), or alternatively, the end of a search window (with a defined period of time before the ending event). In a case where a match region is not defined in relation to a cardiac event, the search window may still need to be defined in relation to a cardiac event.

A value entered for a time period for a search window may be, for example, a positive number of milliseconds that defines the time before and the time after a cardiac event. For example, as will be described in greater detail below in connection with FIG. 8C-8D, marks indicating the end of the T-waves may have been previously placed on all individual ECG cycles in an ECG trace by the attribute analysis module 234. Alternatively, marks indicating the peak of the R-wave may be indicated, with an estimate of where the T-wave would occur indicated by using an estimated distance after the peak of the R-wave (with the estimated distance being the distance from the R-wave to the end of the T-wave in the template). As such, the search window selection module 233 can be used to define a search window that includes one or more of the cardiac markers. When comparisons are made between ECG cycles and templates, the analysis execution module 235 can identify, using the search window information associated with the ECG template, the portions of the individual ECG cycles to use in the comparison. For example, the region on each ECG cycle to be used in the matching can be determined by first finding the peak of the R-wave (and/or the end of the T-wave), and then determining when the region ends and begins in relation to the end of the T-wave.

It will be appreciated that the time length of a match region and a search window need not, and in many cases will not, be the same. When the time lengths are different, in the similarity comparison analysis the match region may be scanned through the search region as described previously. At various points (e.g., starting at each successive sample) in the scan a comparison of the template to the search window at that point will be performed (using only overlapping portions of the match region and the search window); if at any point in the scan the comparison yields a degree of similarity that is above a specified degree, the comparison may be considered to be a match.

In operation 720 of the FIG. 7 method, a specification is made as to a minimum degree of similarity that must be present for an analyzed cycle to be considered to match the match region of the template. A specification of a minimum degree of similarity may be made for each of the match regions being defined for a template. The minimum degree of similarity may be specified as a percentage, for comparison algorithms that express a degree of similarity in percentage terms. For example, a user may select a minimum match percentage, which can be a percentage value between 0% and 100% (e.g., 85%). The minimum match percentage is a parameter that can be used by the analysis execution module 235 as a minimum threshold value for determining what constitutes a match. For example, if a comparison algorithm finds that a portion of an ECG cycle in a template shows a similarity of less than 85% (e.g., 84%, 25%, and the like) to a portion of an ECG trace, and the minimum match percentage is set to 85%, then the algorithm will not consider the two a match. If, however, a comparison yields a similarity of 85% or greater where the minimum match percentage is set at 85%, the algorithm can indicate that a match has been found.

In other comparison algorithms, other measures of similarity besides percentage measures may be used. In addition, the form of similarity measure that is selected by a user may be converted into a form applicable to a particular comparison algorithm being used.

In operation 725, a user may select a maximum match percentage value to be used in the comparison analysis. Again, this may be done for each of the match regions being defined for a template. This maximum threshold is used to indicate the level of a match, after which an individual ECG cycle will no longer be compared to templates. For example, the user may set a maximum match percentage value of 95% for the T-wave region of a single template 225. If a match is found between the T-wave region of an individual ECG cycle and the T-wave region of the template, such that the similarity is less than 95% (e.g., 87%, 35%, and the like), the comparison algorithm may continue to compare the ECG cycle to other match regions and/or templates. However, if the match described yields a similarity which is greater than the 95% value entered (e.g., 97% and the like), the comparison algorithm may no longer compare the individual ECG cycle to additional templates for the purpose of finding matches.

In operation 730, the user may select a normalization percentage relating to amplitude scaling during the comparison process. Again, this may be done for each of the match regions being defined for a template. When a portion of an individual ECG cycle is being compared to a match region of an ECG template, it is possible to scale the amplitude of the ECG cycle and/or the template to maximize the similarity between the two. In some examples, a user may not be concerned with the amplitude differences between the ECG cycle and the template, and may only be concerned with the relative shapes of the two. In these examples, the user may not want to impose restrictions on the amount of amplitude scaling that can be performed during the comparison. However, in other embodiments, the user may restrict the amount of amplitude scaling performed through the use of the normalization limit percentage.

Referring now to FIGS. 8A-8D, there is shown a series of diagrams that illustrate an implementation of the method 700 of FIG. 7. FIG. 8A is a template selection display 650 that may be provided as a “pop up” box that is displayed after a user has selected a cycle of an ECG trace to use as a template. The display 650 includes a column that lists four waves of the ECG cycle, namely, the P-wave, Q-wave, S-wave, and T-wave. The display 650 includes, next to each of the listed waves, a selectable box (user-selectable boxes 660A-660D) that allows a user to select a wave to serve as a match region (see operation 710). In the FIG. 8A example, the user has selected only one wave for a match region, the T-wave. The user may select multiple waves for a match region, and configure each template.

The template selection screen display 650 of FIG. 8A also gives a user the ability to select the degree of similarity (e.g., the “Minimum Match %”) for each of the waves of the cardiac cycle (see operation 720 of FIG. 7). To accomplish this selection, the template selection screen 650 has another column that includes four boxes 670A-670D, one box for each of the waves. One of the boxes 670A-670D is located in a position (row) corresponding to one of the waves. A user may enter a number in the corresponding box for a wave, and the entered number sets a minimum match percentage to be used for a template for the wave (as described above in connection with operation 720 of FIG. 7).

The template selection screen 650 also has, for each of the four waves (match regions), a selectable button (“Advanced . . . ”) to access an advanced setup screen (see FIG. 8B) for the template corresponding to the wave. When a user selects one of these “advanced options” buttons, an additional or different display screen may be provided on the display device that allows the user to set up more detail for the applicable template and how an analysis using that template will be performed. In addition, the template selection screen 650 includes (at the bottom of the display 650) “OK” and “Cancel” user-selectable buttons that enables a user to accept or to cancel information input into the template selection screen 650. In some implementations, selection of the “OK” button causes the information identified in the template selection screen 650 to be stored in memory (for example, in the template configuration storage 224 in the FIG. 1 embodiment), and also causes the template selection screen 650 to stop being displayed.

FIG. 8B shows an advanced setup screen 750 for a T-wave match region, with values having been entered that may be applicable in the analysis of an ECG trace from a dog subject. Before discussing this advanced setup screen 750, there will first be provided a discussion on how match regions and search windows are defined in a system implementation to which the screenshots of FIGS. 8A and 8B and the diagrams of FIGS. 8C and 8D apply. It will be appreciated that the discussion below is an example only, and that template regions and search windows may be defined in a variety of different ways.

In this implementation, a match region for a P-wave is defined in relation to a marker for the beginning of the P-wave (a “P Start” mark) and in relation to a marker for the end of the P-wave (a “P End” mark). An inside amount of time and an outside amount of time are defined to be included within the match region, with the inside amount of time being immediately after the P Start mark (or alternatively before the P End mark), and the outside amount of time being immediately before the P Start mark (or alternatively after the P End mark). In other words, “inside” means the inside of the P-wave, and “outside” means outside the P-wave. The amount of time for the inside and outside amounts of time are defined as percentages of the amount of time between the P Start mark and the P End mark. Thus, for example, an inside time percentage may be defined to be 80%, in which case the inside amount of time would be 80% of the time between the P Start mark and the P end mark. That amount of time before the P End mark (or alternatively after the P Start mark) would be included in the match region. In addition, for example, an outside time percentage may be defined to be 35%, in which case the outside amount of time would be 35% of the time between the P Start mark and the P End mark, and that amount of time after the P End mark (or alternatively before the P Start mark) would be included in the match region.

As for the region of a cycle of an ECG trace to which a P-wave match region is compared (which we will refer to as a comparison region), it is defined by a search window parameter value, which is expressed in milliseconds. In identifying where the comparison region occurs in a cycle, an identified R-wave peak event serves as an anchor that determines the location of the comparison region. The R-wave peak event may be used, as it is in this implementation, because it is the most easily definable attribute in the cycle. Then, the comparison region of the cycle under analysis includes the previously defined match region offset from the detected R-wave in the same direction and distance that the match region is offset in the template from the R-wave mark in the template. In addition, the comparison region includes an additional amount of time at both the beginning and at the end of the comparison region equaling the amount of time of the search window value.

For a Q-wave match region, the match region is defined in relation to the Q mark. The inside and outside amounts of time (that is, the time after the Q mark included in the match region, which is “inside” the QRS complex, and the amount of time before the Q mark that is included in the match region, which is “outside” the QRS complex) are expressed as percentages of the time between the Q and the R Peak marks. The comparison region again uses the peak of the R-wave in the cycle being analyzed as an anchor. From there, the comparison region of the cycle under analysis includes the previously defined match region offset from the detected R-wave in the same direction and distance that the match region is offset in the template from the R-wave mark in the template. In addition, the comparison region includes an additional amount of time at both the beginning and the end of the comparison region equaling the amount of time of the search window value.

For an S-wave match region, the match region is defined in relation to the S mark. The inside and outside amounts of time (that is, the time before the S mark included in the match region, which is “inside” the QRS complex, and the amount of time after the S mark included in the match region, which is “outside” the QRS complex) are expressed as percentages of the time between the R Peak and S marks. The comparison region again uses the peak of the R-wave in the cycle being analyzed as an anchor. From there, the comparison region of the cycle under analysis includes the previously defined match region offset from the detected R-wave in the same direction and distance that the match region is offset in the template from the R-wave mark in the template. In addition, the comparison region includes an additional amount of time at both the beginning and the end of the comparison region equaling the amount of time of the search window value.

Finally, for a T-wave match region, the match region is defined in relation to the T Peak and the T End marks (as with the P-wave match region where the match region is defined in relation to the P Start and P End marks). An inside amount of time and an outside amount of time are defined to be included within the match region, with the inside amount of time being immediately after the T Peak mark (or alternatively before the T End mark), and the outside amount of time being immediately before the T Peak mark (or alternatively after the T Peak mark). In other words, “inside” means, generally at least, the inside of the T-wave. The amount of time for the inside and outside amounts of time are defined as percentages of the amount of time between the T Peak mark and the T End mark. The comparison region of the cycle under analysis includes the previously defined match region offset from the detected R-wave in the same direction and distance that the match region is offset in the template from the R-wave mark in the template. In addition, the comparison region includes an additional amount of time at both the beginning and the end of the comparison region equaling the amount of time of the search window value.

With that background, a discussion of the advanced setup screen for a T-wave template, as shown in FIG. 8A, will now be provided. The advanced setup screen 750 of FIG. 8B may be displayed upon an advanced setup button for the T-wave match region being activated in the template selection screen 650 shown in FIG. 8A. The advanced setup screen 750 includes five options that may be specified by a user. First, the screen 750 includes a search window option, in which a user inputs a time period in milliseconds for the search window. In the FIG. 8B example, the number 40 is shown displayed in box 760 for the search window, which indicates 40 milliseconds for the length of the search window (which may be appropriate in a case of an ECG of a dog subject). As discussed above, this is a length of time for the comparison region in the cycle being analyzed, which will added to both the beginning and the end of a region defined by inside and outside percentage parameters. The number 40 may be a default value that is entered by a user activating a “Load Default” button provided at the bottom of the advanced setup screen 750, thus setting the search window to 40 milliseconds.

The second and third options provided on the advanced setup screen 750 are an “Outside (%)” and an “Inside (%)” with a box (770 and 780) associated with each of these. These options define a starting point and an ending point of the T-wave match region, and also, as described above, are used in identifying the comparison region of a cycle being analyzed. The “Inside (%)” option specifies a period of time after the marked T Peak, or alternatively before the T End mark, that is to be included in the T-wave match region, and the “Outside (%)” option specifies a period of time after the marked T End mark, or alternatively before the T Peak mark, that is to be included in the T-wave match region. The length of these time periods is indicated as a percentage of the amount of time between the T Peak mark and the T End mark in the template.

In the FIG. 8B example, a value of 75 is displayed for the “Outside (%),” and a value of 175 is displayed for the “Inside (%).” As such, the “inside” period of time before the marked T End mark (or alternatively after the T Peak mark) that will be included in the match region is 175% of the amount of time between the T Peak mark and the T End mark of the template. Assuming the amount of time between the T Peak mark and the T End mark is 40 milliseconds, the “inside” amount of time would be 70 milliseconds. The “outside” period of time after the marked T End mark (or alternatively before the marked T End mark) that will to be included in the match region is 75% of 40 milliseconds, or 30 milliseconds. In total, the T-wave match region would thus be a 100 millisecond period defined in relation to where the T Peak (or T End) mark is marked in the template.

Referring now to FIG. 8C, there is shown a graphical illustration of a template trace 810 and a T-wave match region 815 (and specifically for a T End mark match region) whose settings are specified in the setup screen of FIGS. 8A and 8B. An end of a T-wave in the template trace 810 is marked by a “Tend” mark (or “Template Tend”) 820. A portion of the template trace 810 that makes up the T-wave match region is identified and is positioned in time in relation to that Tend mark 820. In particular, the match region includes an “inside” portion 830 of the trace 810 (inside the T wave) and an “outside” portion 840 of the trace 810 (outside the T wave), both portions being anchored in relation to the Tend mark 820 of the trace 810. The inside portion 830 of the match region 815 immediately precedes the Tend mark 820, and the outside portion 840 of the match region 815 is immediately after the Tend mark 820. Based on the settings shown in FIG. 8C and an example of a 40 millisecond period of time between a T Peak mark (not shown in FIG. 8C) and the Tend mark 820 in the template, the inside portion 830 of the match region 815 is 175% of 40 milliseconds, or 70 milliseconds, and the outside portion 840 of the match region 815 is 75% of 40 milliseconds, or 30 milliseconds. As such, the T-wave match region 815, in this example, is a 100 millisecond long portion of the template trace 810 and is positioned within the template trace 810 based on the location of the Tend mark 820.

Referring now to FIG. 8D, there is shown a graphical illustration of the template trace 810 shown in FIG. 8C, and in addition, a trace 850 of a cardiac cycle of an ECG trace being analyzed is shown overlying the template trace 810. The two traces 810 and 850 are aligned in time by peaks 855 of their R-waves. A search window 860 identifies a portion of the trace under analysis 850 to which the template match region 815 (FIG. 8C) is to be compared. The length of the search window 860 is 180 milliseconds, as dictated by the length of the match region 815 (100 milliseconds) and the setting in the search window field 760 of FIG. 8B, which adds a 40 millisecond period 870 to both the beginning and the end of the match region 815. The search window 860 is positioned in time within the trace under analysis 850 such that the point 820 on the trace 850 upon which the inside and outside match regions 830 and 840 are set (which in turn set where the search window periods 870 begin) is offset from the R-wave of the cycle under analysis 850 by a time period equaling an amount of time between the R Peak mark of the template trace 810 and the Tend mark 820 of the template trace 810. The point 870 upon which the position of the search window 860 is set, because the two traces 810 and 850 have the peaks 855 of their R-waves aligned, therefore is shown in FIG. 8D aligned in time with the Tend mark 820 of the template trace 810. Accordingly, in this implementation, a Tend mark 865 that marks an end of the T-wave in the trace under analysis 850 is not used in identifying the location 870 upon which the position of the search window 860 is set. Rather, the location of the search window 860 is anchored on the peak 855 of the R-wave of the trace under analysis 850, although is offset in time from that anchor location by an amount of time equaling the time length between the R Peak mark and the Tend mark in the template trace 810.

Referring back again to FIG. 8B, options are provided on the advanced setup screen 750 for values for the previously described maximum match percentage and normalization limit percentage are provided. In the case of FIG. 8B, a value of 95 is shown for the maximum match percentage, and a value of 50 is shown for the normalization limit percent. If accepted, these are the values that will be used during execution of an analysis using the configured T-wave template. The advanced setup screen 750 also includes a “Load Default” button that may be selected by a user to load in the boxes above the button default values. Finally, the advanced setup screen 750 includes (at the bottom of the display 750) “OK” and “Cancel” user-selectable buttons that enables a user to accept or to cancel information input into the advanced setup screen 750. In some implementations, selection of the “OK” button causes the information identified in the advanced setup screen 750 to be stored in memory (for example, in the template configuration storage 224 in the FIG. 1 embodiment), and also causes the advanced setup screen 750 to stop being displayed.

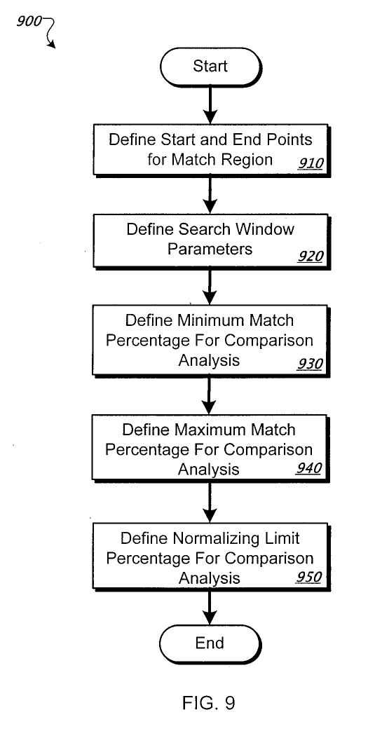

Referring now to FIG. 9, there is an alternative setup method 900 in which match regions are not defined in relation to a marked event, but rather are defined only with starting and ending points. In operation 910, there is defined a starting point and an ending point for a match region of a template. This may be done, for example, by a user being presented on a display device with a display of the template, and by receiving user input that specifies where on the template the match region begins and where the match region ends. This may be done, for example, by a user navigating to an appropriate location on the template with a pointing device such as a mouse, and entering a mouse click to identify a selected location for the beginning and ending points. In operation 920, a search window is defined. In one implementation, the search window may still be defined in relation to an event detected through attribute analysis, even though the match region is not defined in relation to a marked event. In such a case, a user may specify a cardiac event in relation to which the search window will be identified, and then a time period before and/or after the cardiac event to be included in the search window. In operations 930, 940 and 950, there is defined for comparison analyses using the defined template and search window, a minimum match percentage, a maximum match percentage, and a normalizing limit percentage, for example, in the manner previously discussed.

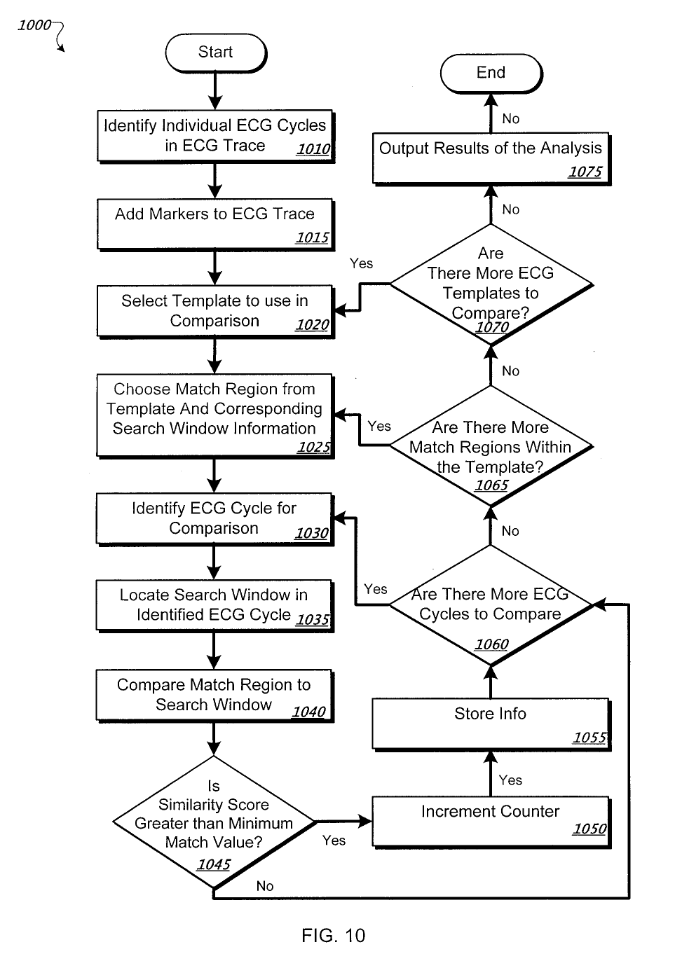

FIG. 10 is a flowchart of an overall analysis process 1000 for analyzing an ECG trace that involves defining multiple different templates and multiple different match regions, and performing comparison analyses using those defined match regions. The process 1000 may be executed by the system 200 shown in FIG. 1, for example. In operation 1010, a received ECG trace is analyzed by one or more attribute-based algorithms included in the attribute analysis module 234 to identify within the ECG trace one or more individual cardiac beat-to-beat cycles, such as the ECG cycles 132. In operation 1015, the attribute analysis module 234 can use markers to label the location of cardiac events such as the P-, Q-, S-, and T-waves within the individual ECG cycles. In operation 1020, the analysis execution module 235 can allow a user to select one of the templates from the template configuration storage 224 to use in a comparison. In operation 1025, the analysis execution module 235 can allow a user to select a match region from a template and obtain the corresponding search window information and/or search parameters (e.g., the minimum search value, the maximum search value, and the like) from the template.

In operation 1030, with an analysis now set up to be executed, execution of a comparison analysis begins. In particular, the analysis execution module 235 can identify one of the individual ECG cycles to use in a comparison. This may be done, for example, by identifying an R-wave peak in a trace under analysis. In operation 1035, the analysis execution module 235 can use the search window information obtained in operation 1025 to determine the search window in the ECG cycle identified in operation 1030. In operation 1040, the analysis execution module 235 can compare the match region selected in operation 1020 to the search window identified in operation 1035, and can determine a similarity score for the comparison analysis. In operation 1045, the similarity score is compared to a minimum similarity value. If the similarity score is greater than the minimum similarity value, operation 1050 is performed, causing a counter associated with the match region of the currently selected template to be incremented. The counter can keep track of the number of individual ECG cycles within an ECG trace that match the match region of the current template.

Performing operation 1055 causes the analysis execution module 235 to store information associated with the match determined in operations 1040 and 1045 in the analysis output results storage 226. Exemplary information that can be stored can include the number of ECG cycles that have been identified as matching the match region of a template, information identifying which match region matched the ECG cycle, and the like. In operation 1060, the analysis execution module 235 determines if there are any additional ECG cycles to compare to the current template match region. If there are more ECG cycles to compare to the match region, the process 1000 can return to 1030, where the analysis execution module 235 can identify another ECG cycle for comparison. If there are no additional ECG cycles to compare to the current match region, the analysis using that match region is complete, and results of the analysis using that match region may be displayed.

In reviewing the results, a user may determine that another match region for the template, and operation 1065 may be performed to determine if there are any additional match regions in the current template to be used in a comparison. Alternatively, in a case where multiple match regions are configured before an analysis is run, the system may make this determination without user involvement. If there are additional match regions to be used for comparison, the process 1000 can return to operation 1025 where the analysis execution module 235 can allow a user to select (or alternatively the system selects) an additional match region from the current template for comparison, and a comparison analysis can be executed as with the first match region, and the result of the comparison analysis may be displayed.

If it is determined at operation 1065 that there are no additional match regions in the template, operation 1070 may be performed in which a user (or alternatively, the system) determines if it would be useful to run an analysis using a different template. If the analysis execution module 235 determines, in operation 1070, that there are more ECG templates to be used in comparisons, the process 1000 can return to operation 1020, where a new template is selected, and various match regions for that template may be analyzed as previously described. If there are no more templates, the process 1000 can perform operation 1075 and output overall results of the analysis to, for example, the display device 250.

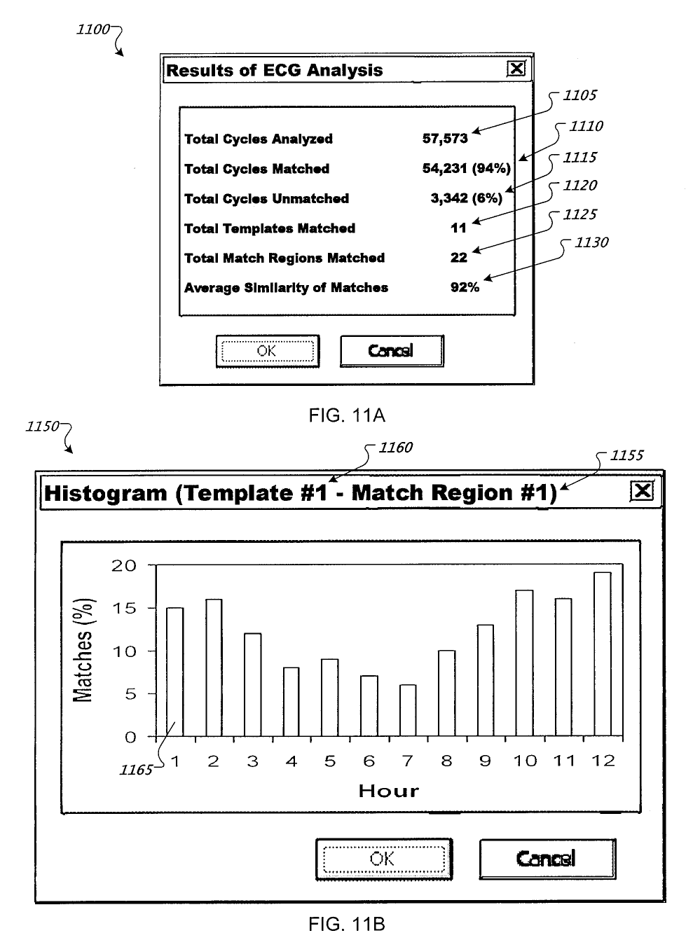

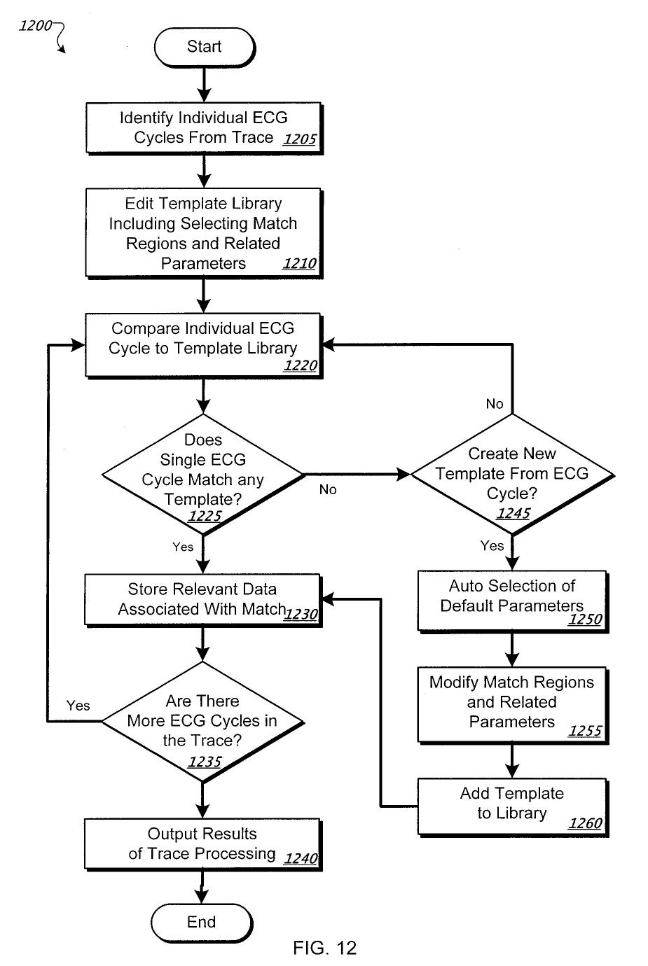

FIGS. 11A and 11B show example displays that may be produced for ECG analyses performed in accordance with the above-described techniques. Referring first to FIG. 11A, numerical information about the number of cycles analyzed and the number of matches may be displayed in the form of a graphical user interface (GUI) 1100. In the example results display shown in FIG. 11A, the total number of cardiac cycles (e.g., 57,573) analyzed in a ECG trace can be displayed to the user as total cycles analyzed 1105. In the example shown in FIG. 11A, the total number of individual ECG cycles that were matched to ECG templates is 54,231 (94% of the total cycles analyzed) and is displayed to the user as total cycles matched 1110. In this example, a total of 3,342 ECG cycles (6% of the total cycles analyzed) were not matched and this information is displayed to the user as total cycles unmatched 1115. During the analysis of an ECG trace, one or more templates can be matched to one or more portions of individual ECG cycles. In the example shown here, eleven of the templates were matched to ECG cycles in the ECG trace. This is displayed as total templates matched 1120.

In some cases, the templates can each include one or more match regions that can be compared to portions of the ECG cycles, thus the FIG. 11A display shows that the total number of match regions that match individual ECG cycles may be different than the total templates matched 1120. In this example, the total number of match regions that match portions of ECG cycles is 22 and is displayed as total match regions matched 1125. When match regions are compared to portions of ECG cycles, a similarity score can be assigned to each comparison indicating the degree of similarity between the match region and the portion of an individual ECG cycle it is compared to. When the similarity score exceeds the minimum match percentage (see the description in connection with FIG. 8A), the comparison is determined a match. At the end of the analysis, the similarity scores of the matches can be averaged and displayed to the user. In the example described here, the average similarity of matches 1130 is 92%.

Referring now to FIG. 11B, a results display in histogram format may also be provided. In FIG. 11B, a portion of the results obtained during an ECG analysis can be displayed to a user in the form of a GUI 1150 on, for example, the display device 250 and/or a desktop computer. The results shown in the FIG. 11B display may be generated after the following example analysis is performed. The ECG trace being analyzed may be for a period of time (e.g., 12 hours). During analysis of the ECG trace, one or more match regions of one or more templates can be compared to the search windows of the individual ECG cycles in the ECG trace. In some embodiments, when a comparison between a match region of a template and a search window of an ECG cycle yields a similarity greater than the minimum match percentage 670 (see FIG. 8A), the comparison is considered a match and data associated with the match can be stored in the analysis output results storage 226. Exemplary data can include the relative time in the ECG trace (e.g., 1 hour 43 minutes 22 seconds from the start of the ECG trace, the first hour, the fifth hour, or the like) that the matched ECG cycle occurred. After completion of the analysis, the time related data can be used to create a histogram such as the one in FIG. 11B. In this example, the GUI 1150 displays a histogram that shows the time distribution of ECG cycles that matched a match region #1 1155 of a template #1 1160. For example, during the first hour of the ECG trace 130 (shown as vertical bar 1165), 15% of the ECG cycles 132 matched the match region #1 1155 of the template #1 1160. While one histogram 1150 is shown here, additional histograms can be displayed for one or more of the other match regions contained in templates used by the analysis execution module 235.

Referring now to FIG. 12, there is shown a flowchart of a process 1200 that may be executed to determine if a cycle of an ECG trace would be an appropriate selection for use as a template. In operation 1205 of the FIG. 12 method, an attribute analysis module 234 can process an ECG trace, received by the ECG analysis software program 210, by separating it into one or more individual ECG cycles. The ECG cycles can each represent the electrical activity associated with one complete cardiac cycle (e.g., all of the cardiac events related to the flow of blood that occur from the beginning of one heartbeat to the beginning of the next). For example, the attribute analysis module 234 can separate the ECG trace into individual ECG cycles by determining the start and stop points of the cardiac cycle based on the occurrence of “R” waves.

In operation 1210, a user can optionally edit a library of templates (e.g., the ECG templates 225 depicted in FIG. 1) that were previously derived, at least in part, from individual ECG cycles. These templates can include information in addition to the ECG cycles themselves, including information related to match regions (described previously in more detail in connection with FIGS. 7 and 8A-8D). Exemplary edits that can be performed include modifying existing match regions, adding additional match regions, removing match regions, and removing templates from the library.

In operation 1220, an individual ECG cycle selected from the ECG trace can be compared by the analysis execution module 235 to the ECG templates contained in the template configuration storage 224. Some embodiments of a process for comparing an ECG trace to stored ECG templates were described previously in connection with FIG. 10. In operation 1225, if it is determined that the selected ECG cycle does match one or more of the existing ECG templates, the process 1200 continues to operation 1230, where information related to the match is stored, for example, in the analysis output results storage 226. Exemplary information related to the match can include the time that the ECG cycle was recorded, a unique identifier for the ECG cycle, one or more numbers related to the degree of matching (e.g., a percentage), and the like. In operation 1235, if there are more ECG cycles to be compared, the process 1200 can return to operation 1220 where an additional ECG cycle can be compared to the ECG templates contained in the template configuration storage 224. If, in operation 1235, it is determined that there are no more remaining-ECG cycles to compare, the process 1200 continues to operation 1240 where the results of the ECG trace analysis can be output to a user, for example, via the display device 250.

Revisiting operation 1225, if the ECG cycle selected in operation 1220 does not match any ECG templates, the user may be given the option, in operation 1245, to create a new ECG template from the selected ECG cycle. If the user chooses not to add a template based on the ECG cycle, the process 1200 can return to operation 1220, where the matching processes can repeat using a different ECG cycle. If the user chooses to add a template based on the new ECG cycle, operation 1250 can be performed and the template modification device 215 can be used to create a new template. In operation 1250, the template device 214 can select default parameters to be used with the new template. For example, the attribute analysis module 234 may have previously identified the Q-, R-, S-, T-, and U-waves in the ECG cycle, the match region selection module 232 may have selected preliminary match regions corresponding to these waves, and the search window selection module 233 may have previously selected search windows corresponding to the match regions.

In operation 1255, the user can be given the opportunity to modify the default parameters selected in operation 1250. For example, the user may select one or more of the Q-, R-, S-, T-, and U-waves to use as match regions. The user may also modify the parameters, such as size, of one or more of the default regions. The process of modifying cardiac markers, search windows, match regions, and associated parameters was described previously in connection with FIGS. 5-8D. When using multiple match regions, the user may assign a “weight” to each region being matched for the purpose of giving each region a different level of priority in the matching. After the parameters have been selected in operation 1255, operation 1260 can be executed causing the ECG template to be saved to the template configuration storage 224. The process 1200 may advance to operation 1230, where match information is stored in the analysis output results storage 226. In this example where the ECG template was derived from the individual ECG cycle it is being matched to, the cycle and the template may be identical, thus yielding a 100% similarity score.

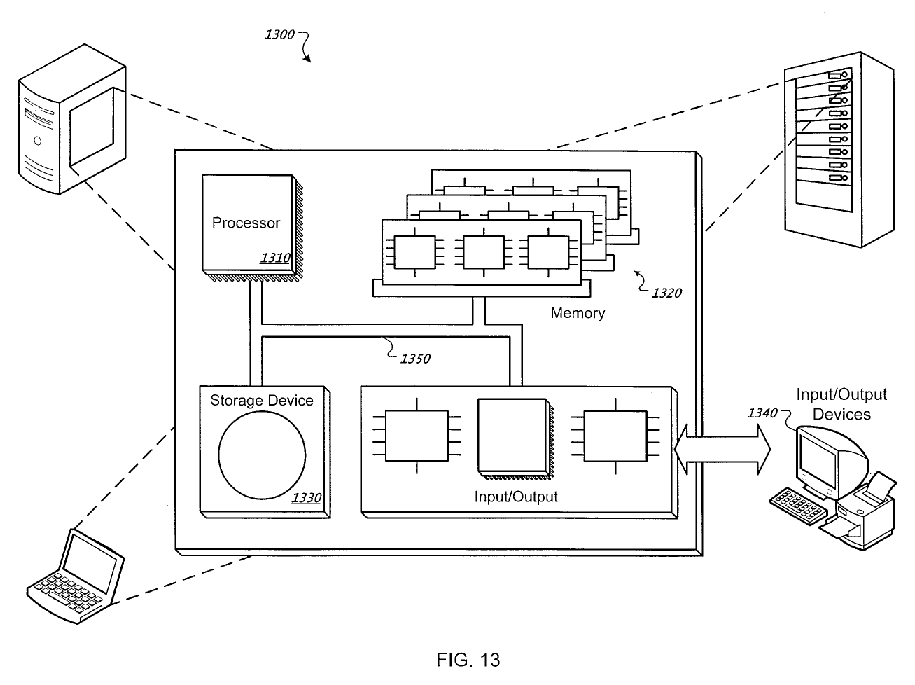

FIG. 13 is a schematic of a general computing system 1300. The system 1300 can be used in carrying out the operations described in association with any of the computer-implemented methods described previously. The system 1300 includes a processor 1310, a memory 1320, a storage device 1330, and an input/output device 1340. Each of the components 1310, 1320, 1330, and 1340 are interconnected using a system bus 1350. The processor 1310 is capable of processing instructions for execution within the system 1300. In one implementation, the processor 1310 is a single-threaded processor. In another implementation, the processor 1310 is a multi-threaded processor. The processor 1310 is capable of processing instructions stored in the memory 1320 or on the storage device 1330 to display graphical information for a user interface on the input/output device 1340.

The memory 1320 stores information within the system 1300. In one implementation, the memory 1320 is a computer-readable medium. In one implementation, the memory 1320 is a volatile memory unit. In another implementation, the memory 1320 is a non-volatile memory unit.

The storage device 1330 is capable of providing mass storage for the system 1300. In one implementation, the storage device 1330 is a computer-readable medium. In various different implementations, the storage device 1330 may be a floppy disk device, a hard disk device, an optical disk device, or a tape device.

The input/output device 1340 provides input/output operations for the system 1300. In one implementation, the input/output device 1340 includes a keyboard and/or pointing device. In another implementation, the input/output device 1340 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device or in a propagated signal, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

A number of embodiments of the invention have been described. Nevertheless, it will be understood that various modifications may be made without departing from the spirit and scope of the invention. Accordingly, other embodiments are within the scope of the following claims.

What is claimed is:

1. A computer-implemented method of analyzing an electrocardiogram (ECG) trace, the method comprising:
defining a match region within an ECG signal template comprising one cardiac cycle, wherein the match region comprises a portion of the ECG signal template that is shorter than the entire template;
performing a similarity comparison analysis on an ECG trace, the similarity comparison analysis comprising, for at least one cardiac cycle within the ECG trace, a comparison of the defined match region to a corresponding region of the cardiac cycle of the ECG trace to determine a degree of similarity between the defined match region and the corresponding region of the cardiac cycle of the ECG trace; and
producing a results output based upon the similarity comparison analysis.

2. The computer-implemented method of claim 1, wherein the defining of the match region comprises receiving user input that defines the match region

3. The computer-implemented method of claim 2, wherein the user input that defines the match region comprises user input that selects a marked cardiac event related to the defined match region.

4. The computer implemented-method of claim 3, wherein the user input that defines the match region further comprises user input that selects a period of time in relation to the marked cardiac event.

5. The computer implemented-method of claim 1, wherein the defining of the match region comprises receiving user input that selects a starting point and an ending point for the match region.

6. The computer implemented-method of claim 1, wherein:
the defining of the match region comprises defining a first match region and a second match region that is different from the first match region;
the performing of the similarity comparison analysis on the ECG trace comprises performing the similarity comparison analysis a first time using the first defined match region and performing the similarity comparison analysis a second time using the second defined match region.

7. The computer implemented-method of claim 1, wherein the output is provided on a visual display device.

8. The computer-implemented method of claim 1, wherein the results output comprises a total number of cardiac cycles processed in the similarity comparison analysis, and a total number of the processed cardiac cycles for which the corresponding region of the cardiac cycle was determined to have the specified degree of similarity to the match region.

9. The computer-implemented method of claim 1, wherein the results output comprises a time distribution of when there occurred, in the ECG trace, cardiac cycles for which the corresponding region of the cardiac cycle was determined to have the specified degree of similarity to the match region.

10. The computer-implemented method of claim 9, wherein the results output comprises histogram formatted results for each of multiple different time periods within the period of time during which the ECG trace was obtained.

11. The computer-implemented method of claim 10, wherein the results output comprises, for each of the multiple different time periods, a total number of cycles processed in the similarity comparison analysis within the time period, and a total number of the processed cardiac cycles within the time period for which the corresponding region of the cardiac cycle was determined to have the specified degree of similarity to the match region.

12. The computer-implemented method of claim 11, wherein the output with the results is provided on a visual display device.

13. The computer-implemented method of claim 1, further comprising defining search window parameters used to identify the corresponding region of the cardiac cycle of the ECG trace that is compared to the match region.

14. The computer-implemented method of claim 13, wherein the search window parameters comprises a specified cardiac event that is identified in the cardiac cycle by attribute analysis.

15. The computer-implemented method of claim 14, wherein the search window parameters further comprise a time period defined in relation to the cardiac event.

16. The computer-implemented method of claim 1, further comprising specifying which of the cardiac cycles within the ECG trace are to be included in the similarity comparison analysis.

17. The computer-implemented method of claim 1, wherein the ECG trace is obtained from a subcutaneously implanted device that obtains a subcutaneous ECG trace and telemeters digital data comprising the subcutaneous ECG trace to external equipment for processing.

18. The computer-implemented method of claim 1, wherein the ECG trace is obtained using surface electrodes place on an external surface of a subject.

19. The computer-implemented method of claim 1, further comprising defining a cycle of the ECG trace to serve as the ECG signal template.

20. The computer-implemented method of claim 1, wherein the ECG signal template is a cardiac cycle taken from an ECG trace that is different from the ECG trace on which the similarity comparison analysis is performed.

21. A computer-implemented method of analyzing a signal trace, the method comprising:
defining a match region within a signal template, wherein the match region comprises a portion of the template that is shorter than the entire template;
performing a similarity comparison analysis on the signal trace, the similarity comparison analysis comprising, for at least a portion of the trace having a time span that corresponds with the signal template, a comparison of the defined match region of the template to a corresponding region of the portion of the signal trace to determine a degree of similarity between the defined match region and the corresponding region of the signal trace; and
producing a results output based upon the similarity comparison analysis.

22. The computer-implemented method of claim 21, wherein the signal trace is an electrocardiogram (ECG) signal trace, and the signal template is a cardiac cycle of an ECG signal trace.

23. The computer-implemented method of claim 21, wherein the signal trace is an action potential trace.

24. The computer-implemented method of claim 21, wherein the signal trace is an electrogram trace.

25. The computer-implemented method of claim 21, further comprising defining search window parameters used to identify the corresponding region of the portion of the signal trace that is compared to the match region.

26. A computer-implemented method of analyzing an electrocardiogram (ECG) trace, the method comprising:
defining a match region within an ECG signal template comprising one cardiac cycle, wherein the match region comprises a portion of the ECG signal template that is shorter than the entire template;
defining search window parameters used to identify a comparison region within one cardiac cycle of an ECG trace, the comparison region comprising a portion of the one cardiac cycle that is shorter than the entire one cardiac cycle;
performing a similarity comparison analysis on an ECG trace, the similarity comparison analysis comprising, for at least one cardiac cycle within the ECG trace, a comparison of the defined match region to the comparison region of the cardiac cycle of the ECG trace to determine a degree of similarity between the defined match region and the comparison region of the cardiac cycle of the ECG trace; and
producing a results output based upon the similarity comparison analysis.

27. A computer program product tangibly embodied in computer storage medium and comprising software instructions that when executed by a processor perform operations for analyzing an electrocardiogram (ECG) trace, the operations comprising:
defining a match region within an ECG signal template comprising one cardiac cycle, wherein the match region comprises a portion of the ECG signal template that is shorter than the entire template;
performing a similarity comparison analysis on an ECG trace, the similarity comparison analysis comprising, for at least one cardiac cycle within the ECG trace, a comparison of the defined match region to a corresponding region of the cardiac cycle of the ECG trace to determine if there is a specified degree of similarity between the defined match region and the corresponding region of the cardiac cycle of the ECG trace; and
producing a results output of the similarity comparison analysis.

28. A computing system for obtaining and processing an electrocardiogram (ECG) trace, the system comprising:
a match region definition component that defines a match region within an ECG signal template comprising one cardiac cycle, wherein the match region comprises a portion of the ECG signal template that is shorter than the entire template;
a similarity comparison analysis component that performs a similarity comparison analysis on an ECG trace, the similarity comparison analysis comprising, for at least one cardiac cycle within the ECG trace, a comparison of the defined match region to a corresponding region of the cardiac cycle of the ECG trace to determine if there is a specified degree of similarity between the defined match region and the corresponding region of the cardiac cycle of the ECG trace; and
an output device to which a results output of the similarity comparison analysis is provided.
